(19) United States
(12) Patent Application Publication
(10) Pub. No.: US 2006/0167021 A1
(43) Pub. Date: Jul. 27, 2006

(54) Inhibition of src for treatment of reperfusion injury related to revascularization

(75) Inventor: Douglas W. Losordo, Winchester, MA (US)

Correspondence Address: EDWARDS & ANGELL, LLP, P.O. BOX 55874, BOSTON, MA, 02205, US

(73) Assignee: Caritas St. Elizabeth's Medical Center of Boston, Inc., Boston, MA (US)

(21) Appl. No.: 10/530,038

(22) PCT Filed: Oct. 3, 2003
(86) PCT No.: PCT/US03/31430

§ 371 Date: Apr. 1, 2005

Related U.S. Application Data
(60) Provisional application No. 60/416,334, filed on Oct. 4, 2002.

(51) Int. Cl. A61K 31/519; A61K 31/47

(52) U.S. Cl. 514/262.1; 514/313

(57) ABSTRACT

The present invention provides methods for treating, preventing, or reducing reperfusion injury or post-pump syndrome by administering an inhibitor of vascular endothelial growth factor-mediated vascular permeability.

### CROSS-REFERENCE TO RELATED APPLICATION

The present invention claims priority of U.S. Provisional Application 60/416,334, filed Oct. 4, 2002, the disclosure of which is incorporated herein by reference.

### GOVERNMENT GRANTS

At least part of the work contained in this application was performed under government grant HL63414 from the National Institutes of Health. The government may have certain rights in this invention.

### FIELD OF THE INVENTION

The present invention provides methods, including treatment methods, for significantly reducing reperfusion injury by inhibiting Src, thus enhancing recovery from myocardial infarction and revascularization procedures. The methods provided are useful for treatment of ischemia/reperfusion injuries and are useful prophylactically in revascularization procedures, including percutaneous coronary revascularization procedures (e.g., angioplasty, stent, atherectomy, cutting balloon, drug eluting stent, and rotational atherectomy) and surgical coronary revascularization procedures (e.g., bypass surgery), treatments for stroke, and surgical procedures to relieve compartment syndrome.

### BACKGROUND OF THE INVENTION

During myocardial infarction (“MI”), the entire myocardium experiences decreased flow due in part to edema resulting in response to the onset of ischemic injury. Similarly, the ischemia/reperfusion of unstable angina and of percutaneous and surgical revascularization procedures is known to cause myocardial injury or “post-pump syndrome,” for example, in patients who have undergone bypass surgery or any procedure in which cardioplegia is involved. Patients suffering from “post-pump syndrome” generally exhibit a worsening of symptoms following surgery due to ischemia/reperfusion.

A similar situation occurs in patients experiencing “compartment syndrome.” “Compartment syndrome” is a devastating complication of revascularization of ischemic limbs, which involves edema of the tissue and leads to necrosis due to decreased perfusion. For example, vascular blockage or injury disrupting the blood supply can cause edema of the muscle, which is prevented from expanding beyond the limits of the surrounding fascia, resulting in an increase in tissue pressure and a decrease in perfusion, which ultimately leads to necrosis of the muscle.

Another similar situation of ischemia/reperfusion arises in patients suffering from a stroke, cerebrovascular disease, or cerebrovascular accident.

Tissue perfusion is a measure of oxygenated blood reaching the given tissue due to the patency of an artery and the flow of blood in an artery. Tissue vascularization may be disrupted due to blockage, or alternatively, it may result from the loss of blood flow resulting from blood vessel leakage or hemorrhage upstream of the affected site. The deficit in tissue perfusion during acute myocardial infarction, cerebral stroke, surgical revascularization procedures, and other conditions in which tissue vascularization has been disrupted, is a crucial factor in outcome of the patient's condition.

A deficit in tissue perfusion leads to persistent post-ischemic vasogenic edema, which develops as a result of increased vascular permeability (VP). Edema can cause various types of damage including vessel collapse and impaired electrical function, particularly in the heart. Subsequent reperfusion, however, can also cause similar damage in some patients, leading to a treatment paradox. While it is necessary, to unblock an occluded blood vessel or to repair or replace a damaged blood vessel, the ensuing reperfusion can, in some cases, lead to further damage. Likewise, during bypass surgery, it is necessary to stop the heart from beating and to have the patient hooked to a heart pump. Some patients who undergo bypass surgery, for example, may actually experience a worsening of condition (“post-pump syndrome”), which may be the result of ischemia during cessation of cardiac function during surgery.

An arterial blockage may cause a reduction in the flow of blood, but even after the blockage is removed and the artery is opened, if tissue reperfusion fails to occur, further tissue damage may result. For example, disruption of a clot may trigger a chain of events leading to loss of tissue perfusion, rather than a gain of perfusion. One method for measuring VP is the Miles permeability assay (Miles et al., J. Physiol. 118:228-257 (1952); van der Zee et al., Circulation 95: 1030-1037 (1997)).

Historically, treatment of diseases and conditions involving vascular occlusion has focused on the alleviation of the blockage or on reducing tissue damage during the procedure itself.

At present, the ad hoc use of agents, such as nitroglycerine, nitroprusside, adenosine, and verapamil, is used, frequently via intracoronary methods, to augment flow in infarct arteries or in arteries with slow flow after revascularization. These treatments do not work particularly well, as they do not target the underlying pathophysiology. For example, they have never been shown to reduce infarct size, and they have side effects, such as hypotension.

Vascular endothelial growth factor (VEGF) is an endothelial mitogen, which is expressed within hours following ischemic injury, and is a potent mediator of VP. Src family kinases (“SFKs”), a family of nonreceptor protein tyrosine kinases, mediate signaling activity in response to various growth factors, including VEGF. SFKs include an oncogenic protein (v-Src) and the proteins Src (pp60^{src}) (the cellular homolog of v-Src), Fyn (pp59^{c-fyn}), and Yes (pp62^{c-yes}). Other family members include Lyn, Lck, Hck, Ffr, and Blk. Family members control a wide range of downstream signaling events, often via redundant mechanisms. In some instances, other family members may compensate for decreased activity or inactivity of a mutant or absent family member. SFKs play a wide variety of roles in cell cycle control (e.g., lymphokine-mediated cell survival), cell adhesion and movement (e.g., via integrins), and cell proliferation and differentiation (e.g., regulation of VEGF-induced angiogenesis and MAP kinases).

Inhibition of Src by PP1 has recently been shown to reduce ischemia and brain damage after stroke (Paul et al., Nature Medicine 7(2):222-227 (2001)). Ischemia and ensuing brain damage are associated with VP, which is mediated by VEGF. Infarct volumes are reduced in Src −/− knockout mice, as compared to wild-type control and Fyn −/− mice. Src kinase is required during VEGF-induced vascular permeability, and suppression of Src activity decreases VP, minimizing brain injury following stroke.

It would be useful to have methods for reducing VP in patients, who are suffering from MI, unstable angina, compartment syndrome, or other conditions involving disruption of vascularization, or who are undergoing percutaneous or surgical revascularization procedures.

### SUMMARY OF THE INVENTION

In one aspect, the invention provides a method for treating, preventing, or reducing reperfusion injury or post-pump syndrome by administering an inhibitor of vascular endothelial growth factor-mediated vascular permeability.

In another aspect, the invention provides a method for treating, preventing, or reducing reperfusion injury following ischemia, wherein the ischemia is caused by blockage or leakage of a blood vessel, by administering an inhibitor of vascular endothelial growth factor-mediated vascular permeability, wherein

a. the inhibitor comprises an inhibitor of a Src family kinase; and

b. the ischemia is the result of:
i. myocardial infarction;
ii. stroke;
iii. compartment syndrome;
iv. post-pump syndrome; or
v. angina.

In yet another aspect, the invention provides a method for treating, preventing, or reducing injury following bypass surgery by administering an inhibitor of vascular endothelial growth factor-mediated vascular permeability, wherein the inhibitor comprises an inhibitor of a Src family kinase.

In yet another aspect, the invention provides a method for treating, preventing, or reducing reperfusion injury following compartment syndrome by administering an inhibitor of vascular endothelial growth factor-mediated vascular permeability, wherein the inhibitor comprises an inhibitor of a Src family kinase.

### BRIEF DESCRIPTION OF THE DRAWINGS

FIG. 1A is a photograph showing cardiac tissue from control rats 24 hours after induction of MI.

FIG. 1B is a photograph showing cardiac tissue from PPb-treated rats 24 hours after induction of MI.

FIG. 2A is a photograph showing the results of an immunohistochemistry assay for VEGF on control rats hearts 24 hours after induction of MI.

FIG. 2B is a photograph showing the results of an immunohistochemistry assay for VEGF on PPb-treated rats 24 hours after induction of MI.

FIG. 3 is a schematic of the protocol used to measure the dose-dependent effect of PP1 on infarct size.

FIG. 4 is a graph showing dose-dependent reduction of MI size by PP1.

FIG. 5 is a graph showing the dose-dependent effects of Src deficiency and blockade on myocardial ischemia in a murine model.

FIG. 6 is a schematic of the protocol used to measure the PP1-dependent decrease of infact size six hours after ischemia.

FIG. 7 is a graph showing the effects the timing of PP1 administration with respect to Src deficiency and blockade on myocardial ischemia in a murine model.

FIG. 8 is a graph showing the effects of PP1 treatment resulting in reduced infarct size accompanied by decreased myocardial water content.

FIG. 9 is a photograph of in vivo magnetic resonance imaging showing the reduction in volume of edematous tissue.

FIG. 10 is a graph showing the four-week survival rate for PPb-treated (1.5 mg/kg) and control mice.

FIG. 11 is a graph showing the results of echocardiography testing on PP1-treated and control rats (4 weeks post-operative).

FIG. 12 is a schematic of the protocol used to measure the PP1-dependent decrease of ischemia/reperfusion in rats during a 24-hour period.

FIG. 13 is a comparison of two graphs showing the results of echocardiography testing on PP1-treated and control rats (fractional shortening).

FIG. 14 is a comparison of two graphs showing the results of Evan's blue and TTC-staining on PP1-treated and control rats (% infarct size).

FIG. 15 is a graph showing dose-dependent reduction of MI size by PP1.

FIG. 16 is a graph showing dose-dependent reduction of MI size by PP1.

FIG. 17 is a graph showing showing the effects the timing of PP1 administration with respect to Src deficiency and blockade on myocardial ischemia in a murine model.

FIG. 18 is a comparison of two graphs showing the results of echocardiography testing on PP1-treated and control rats (fractional shortening) and the results of Evan's blue and TTC-staining on treated and control rats (% infarct size).

FIG. 19 is a graph showing the results of Evan's blue and TTC-staining on SKI-606 treated and control rats (% infarct size).

FIGS. 20A-20D are immunoblots showing the results of a series of immunoprecipitations and immunoblotting studies of the Flk-cadherin-catenin complex.

FIG. 21A is a graph comparing the % myocardial water content of wild-type vs. pp60src −/− mutant mice.

FIG. 21B is a graph comparing the % infarct size of wild-type vs. pp60src −/− mutant mice.

FIG. 22A is a series of MRI T2 maps overlayed on gradient echo images in control (top) and PP1 Src inhibitor (bottom) treated rats.

FIG. 22B is a graph showing significant differences of the percentage of LV with T2>40 ms between control, PP1 treated, and SKI-606 treated rats.

### DETAILED DESCRIPTION OF THE INVENTION

VEGF is an endothelial mitogen and a potent mediator of VP. SFKs mediate signaling activity in response to various growth factors, including VEGF. SFKs include an oncogenic protein (v-Src) and the proteins Src (pp60^{c-src}) (the cellular homolog of v-Src), Fyn (pp59^{c-fyn}), and Yes (pp62^{c-yes}). Other family members include Lyn, Lck, Hck, Ffr, and Blk. Family members control a wide range of downstream signaling events, often via redundant mechanisms. In some instances, other family members may compensate for decreased activity or inactivity of a mutant or absent family member.

A “Src family kinase” is a member of the Src family (a Src-related protein) that acts as a kinase (a phosphoryl transfer enzyme utilizing ATP to add a phosphoryl group to a metabolite). Some examples of Src family kinases include, but are not limited to, Src, Fyn, Yes, Lyn, Lck, and Hck.

An “inhibitor” is a substance that reduces an enzyme's activity, for example, by combining with it in a way that influences the binding of substrate and/or its turnover number.

Different Src inhibitors have different activity profiles, inhibit different members of Src family, and may have different side effect profiles. Changes in the chemical composition of Src inhibitors could improve the features of these inhibitors.

Inhibitors of Src include pyrazolopyrimidins, e.g., “PP1” (C₁₆H₁₉N₅, molecular weight 281.4 (BIOMOL Research Laboratories, Inc.; Pfizer) and “PP2.” PP1 inhibits the three SRK isoforms, Src, Fyn, and Yes. PP1 inhibits the enzymatic activity of Lck, Lyn, and Src at IC₅₀ of 5, 6, and 170 nM (Hanke et al. J. Biol. Chem. 271: 695-701 (1996)). In the Examples below, PP1 was used at 0.5-3 mg/kg, equivalent to 22-133 nM for a mouse blood volume of 2 ml.

Another inhibitor of Src is SKI-606 (Wyeth-Ayerst Research), which inhibits Src at 1.2 nM. SKI-606 was used at 0.5-5 mg/kg, equivalent to 12-118 nM in the mouse. “SKI-606,” a 4-anilino-3-quinolinecarbonitrile, is a dual Src/Abl kinase inhibitor with potent antiproliferative activity against CML cells in culture. Treatment with SKI-606 reduces phosphorylation of the autoactivation site of the Src family kinases Lyn and/or Hck.

“Src inhibitors” can act by inhibiting VEGF, preferably as inhibitors of “vascular endothelial growth factor-mediated vascular permeability.” “VEGF-mediated vascular permeability” refers to the permeability of the blood vessels as affected by the activity of VEGF. This characteristic can be measured using the Miles perfusion assay described below. Effects of treatments using the present invention can also be assessed using the Miles perfusion assay.

Administration of the Src or VEGF inhibitor in accordance with the invention can be via injection, e.g., intraperitoneal or intravenous injection. (In embodiments in which the agent is an amino acid sequence, such sequences are preferably produced synthetically or from mammalian cells or other suitable cells and purified prior to use to be essentially or completely free of pyrogens.) The optimal dose for a given therapeutic application can be determined by conventional means and will generally vary depending on a number of factors including the route of administration, the patient's weight, general health, sex, and other such factors recognized by the art-skilled including the extent (or lack) of cell proliferation and/or cycling desired to address a particular medical indication.

Administration can be in a single dose, or a series of doses separated by intervals of days or weeks. The term “single dose” as used herein can be a solitary dose, and can also be a sustained release dose. The subject can be a mammal (e.g, a human or livestock such as cattle and pets such as dogs and cats) and include treatment as a pharmaceutical composition which comprises one or a combination of Src or VEGF modulating agents. Such pharmaceutical compositions of the invention are prepared and used in accordance with procedures known in the art. For example, formulations containing a therapeutically effective amount of one Src or VEGF modulating agent may be presented in unit-dose or multi-dose containers, e.g., sealed ampules and vials, and may be stored in a freeze dried (lyophilized) condition requiring only the addition of the sterile liquid carrier, e.g. water injections, immediately prior use.

For instance, administration of at least one Src or VEGF modulating agent according to the invention can be in amounts ranging between about 1 pg/gram body weight to 100 mg/gram body weight. Precise routes and amounts of administration will vary according to intended use and parameters already discussed.

The present invention provides methods, including treatment methods, for significantly reducing reperfusion and post-pump syndrome injury by inhibiting Src, thus enhancing recovery from myocardial infarction, stroke, compartment syndrome, revascularization procedures and similar conditions. The methods provided are useful for preventing, reducing or treating ischemic chest pain, including myocardial infarction and unstable angina, and are useful prophylactically in coronary revascularization procedures, including percutaneous (e.g., angioplasty, stent, atherectomy, cutting balloon, drug eluting stent, and rotational atherectomy) and surgical (e.g., bypass surgery) procedures; in preventing, reducing, or treating compartment syndrome (e.g., in the extremities); and in preventing, reducing, or treating cerbrovascular reperfusion injury (e.g., following stroke).

Miles Assay

In addition to the methods of the examples below, the effects of treatments using the present invention can also be assessed using the Miles perfusion assay (Miles A A, Miles E M: Vascular reactions to histamine, histamine liberators or leukotoxins in the skin of the guinea pig. J Physiol 1952;118:228-257).

In one example (van der Zee R, Murohara T, Luo Z, Zollmann F, Passeri J, Lekutat C, Isner J M: Vascular endothelial growth factor (VEGF)/vascular permeability factor (VPF) augments nitric oxide release from quiescent rabbit and human vascular endothelium. Circulation 1997;95:1030-1037), male hairless albino guinea pigs (200-400 g) (Charles River Laboratories), which are euthymic and immunocompetent, were lightly anesthetized with ether (Fisher Scientific) and 0.5 ml of a 0.5% (in saline) Evans blue dye solution (Sigma) was injected into the left femoral vein after filtering (0.2 μm micro-pore filter, Corning). 20 min. later indicated reagents were applied by intradermal injection with a 30 gauge needle (Becton Dickinson) causing a bleb of 9-11 mm in diameter. Increase in vascular permeability was assessed by the leakage of blue dye into the bleb. As originally described, a small area of traumatic blueing 1-3 mm in diameter may be seen at the center of the bleb following intradermal injection of saline. The site of intradermal injection was photographed 10 minutes after injection in all animals.

This assay is readily adaptable for the testing of SFK inhibitors to be used as treatments according to the present invention.

In one aspect, the invention provides a method for treating, preventing, or reducing reperfusion injury or post-pump syndrome by administering an inhibitor of vascular endothelial growth factor-mediated vascular permeability.

In a preferred embodiment, the inhibitor comprises an inhibitor of a Src family kinase. In a more preferred embodiment, the Src family kinase comprises Src, Fyn, Yes, Lyn, Lck, or Hck.

Preferably, the inhibitor comprises a pyrazolopyrimidin, more preferably PP1 or PP2. Preferably, the inhibitor has the chemical formula C₁₆H₁₉N₅.

Preferably, the inhibitor comprises a quinolinecarbonitrile. More preferably, the inhibitor comprises a 3-quinolinecarbonitrile, such as a 4-phenylamino-3-quinolinecarbonitrile or a 4-anilino-3-quinolinecarbonitrile. Still more preferably the inhibitor comprises a 4-anilino-3-quinolinecarbonitrile. Still more preferably, the inhibitor comprises SKI-606.

In a preferred embodiment, the inhibitor is administered intravenously.

In other preferred embodiments, the inhibitor is administered by intraperitoneal injection or using an intracoronary method, or is administered percutaneously.

In a preferred embodiment, the method is used to treat a reperfusion injury, wherein the reperfusion injury is the result of myocardial infarction, angina, post-pump syndrome as the result of a coronary revascularization procedure.

In a preferred embodiment, the coronary revascularization procedure comprises a percutaneous coronary revascularization procedure, more preferably comprising angioplasty, stent placement, or atherectomy.

In a preferred embodiment, the coronary revascularization procedure comprises angioplasty, comprising an angioplasty balloon, wherein the balloon comprises a coating comprising an inhibitor of vascular endothelial growth factor-mediated vascular permeability. More preferably, the inhibitor comprises an inhibitor of a Src family kinase.

In a preferred embodiment, the coronary revascularization procedure comprises angioplasty, comprising an angioplasty balloon, wherein the angioplasty balloon is capable of eluting an inhibitor of vascular endothelial growth factor-mediated vascular permeability. More preferably, the inhibitor comprises an inhibitor of a Src family kinase.

In a preferred embodiment, the coronary revascularization procedure comprises stent placement, wherein the stent comprises a coating comprising an inhibitor of vascular endothelial growth factor-mediated vascular permeability. More preferably, the inhibitor comprises an inhibitor of a Src family kinase.

In a preferred embodiment, the coronary revascularization procedure comprises stent placement, wherein the stent is capable of eluting an inhibitor of vascular endothelial growth factor-mediated vascular permeability. More preferably, the inhibitor comprises an inhibitor of a Src family kinase.

In a preferred embodiment, the coronary revascularization procedure comprises a surgical coronary revascularization procedure. More preferably, the surgical coronary revascularization procedure comprises bypass surgery.

In another preferred embodiment, the reperfusion injury is the result of stroke or a treatment for stroke.

In yet another preferred embodiment, the reperfusion injury is the result of compartment syndrome or a treatment for compartment syndrome.

In another aspect, the invention provides a method for treating, preventing, or reducing reperfusion injury following ischemia, wherein the ischemia is caused by blockage or leakage of a blood vessel, by administering an inhibitor of vascular endothelial growth factor-mediated vascular permeability, wherein

a. the inhibitor comprises an inhibitor of a Src family kinase; and

b. the ischemia is the result of:
i. myocardial infarction;
ii. stroke;
iii. compartment syndrome;
iv. post-pump syndrome, or
v. angina.

More preferably, the Src family kinase comprises Src, Fyn, or Yes.

In preferred embodiments, the inhibitor comprises a pyrazolopyrimidin or a 3-quinolinecarbonitrile.

In more preferred embodiments, the inhibitor comprises PP1, PP2, or SK1-606.

In more preferred embodiments, the inhibitor is administered by intravenous, by intraperitoneal injection, by direct injection into an artery, by infusion (either direct or indirect), by an intracoronary method, or by percutaneous administration. Still more preferably, the inhibitor is administered intravenously.

In yet another aspect, the invention provides a method for treating, preventing, or reducing injury following bypass surgery by administering an inhibitor of vascular endothelial growth factor-mediated vascular permeability, wherein the inhibitor comprises an inhibitor of a Src family kinase. More preferably, the inhibitor is administered as part of the cardioplegia solution.

The cardioplegia solution, preferably a high potassium solution, inhibits the heart from beating during bypass surgery, when a pump is used. In a more preferred embodiment, the inhibitor is mixed with the cardioplegia solution. In a still more preferred embodiment, the inhibitor is mixed with a high potassium cardioplegia solution.

In yet another aspect, the invention provides a method for treating, preventing, or reducing reperfusion injury following compartnent syndrome by administering an inhibitor of vascular endothelial growth factor-mediated vascular permeability, wherein the inhibitor comprises an inhibitor of a Src family kinase. More preferably, the inhibitor is administered by infusion into a local artery during a surgical procedure for the treatment or relief of the compartment syndrome.

According to the present invention, the extent of myocardial damage following coronary artery occlusion may be significantly reduced by acute pharmacological blockade of Src kinase.

The following examples are illustrative and are not intended to define the limits of the present invention.

### EXAMPLES

### Cardioprotection by Blockade of Src Activity in Models of Acute Myocardial Infarction

Generally, myocardial Infarction (MI) was induced by ligating the left anterior descending (LAD) coronary artery in Sprague-Dawley rats or in C57 black mice. Intraperitoneal injections of the inhibitors were delivered after the induction of infarction. High resolution magnetic resonance imaging (MRI), dry weight measurements, infarct size, heart volume and area at risk were determined 24 hours after induction of MI. Survival rates and echocardiography were determined at 4 weeks post-MI.

Methods

SFK Inhibitors.

PP1 (BIOMOL Research Laboratories, Inc.) was used at 0.5-3 mg/kg, equivalent to 22-133 nM for a mouse blood volume of 2 ml. SKI-606 (Wyeth-Ayerst Research) was used at 0.5-5 mg/kg, equivalent to 12-118 nM in the mouse.

Ischemic Models.

For the analysis of infarct size, myocardial water content, magnetic resonance imaging, echocardiographic functional and fibrotic tissue experiments, a rat model of acute MI was used. 2-year-old C57/ByJ mice were used as a model of severe MI to test the effects of Src inhibition on survival. The effect of Src inhibition on infarct size was also determined using a rat ischemia/reperfusion model with temporary LAD occlusion for 60 (SKI-606) or 45 minutes (PP1), test agent administered 60 minutes later, and infarct size determined 24 hours later. Adult male Sprague-Dawley rats (Harlan, Indianapolis, Ind.) and C57/ByJ mice (Jackson Laboratory, Bar Harbor, Me.) were maintained under approved protocols.

Infarct Size.

After 24 hours, 10% Evans blue (Sigma, St. Louis, Mo.) was injected intravenously before sacrifice. Hearts were removed and cut in three equivalent sections distal to the occluding LAD suture and one proximal. The distal sections were digitized to evaluate the nonperfused area at risk using NIH Image software. Sections were stained with 2% triphenyltetrazolium chloride (TIC) (Sigma, St. Louis, Mo.) to delineate ischemic area. Infarct size is presented as the percentage of area at risk to eliminate variability. For example, the area at risk (AAR) is measured as a function of (white+red area)/(blue+white+red area); the % infarct is measured as a function of (white area (% of LV area))/(blue+white+red area); and the % infarct/AAR is calculated as a function of (white area (% of AAR))/(white+red area).

Water Content and Cardiac Function.

In-vivo water content was evaluated using MRI performed serially on anesthetized rats 24 hours following MI using a 4.7-T MR scanner (Bruker Billerica Mass.). Src inhibitor treated rats were administered either PP1 (5.0 mg/kg, intraperitoneal, n=2) or SKI-606 (5.0 mg/kg, intravenous, n=5) 45 minutes following permanent LAD occlusion. MRI experiments to quantify T2 values of the myocardium were conducted by applying an ECG and respiratory-triggered multiecho spin echo sequence (number of echoes, 8; echo time, 6.6 ms; slice thickness, 1.0 mm; inplane resolution, 430 μm²; total slices, 6-7). The trigger delay was chosen to capture all echoes during full diastole to avoid motion artifact between echoes. Corresponding gradient echo images were also acquired for each slice to clearly delineate the blood/myocardium border for region of interest evaluation of the spin echo sequence. Because of their increased water content, edematous regions are expected to have a longer T2 relaxation than nonedematous regions. Regions with T2>40 ms (two standard deviations above the mean of normally perfused myocardium) were delineated and the volume calculated as a percentage of the total LV myocardial volume. In addition, ex-vivo myocardial water content of proximal heart sections was measured as the percentage difference between initial wet and dry weights after 24 hours incubation at 80° C. Transthoracic echocardiography (SONOS 5500, Agilent Technologies, Palo Alto, Calif.) was performed to evaluate LV function before (baseline) and 4 weeks after MI. For this analysis, rats were anesthetized with 0.6 ml/kg ketamine intraperitoneally.

Fibrotic Tissue.

For the histopathological analysis of fibrotic tissue, hearts were removed after functional analysis and volume and circumference of fibrotic tissue was determined by staining with elastic trichrome and performing computer-based planimetry. The amount of fibrotic tissue was measured as the percentage of LV area, as well as the percentage of LV circumference, to eliminate the contribution of differences in end diastolic diameter and hypertrophy among the groups.

In vivo Permeability Model.

Adult mice were injected i.v. with 50 μl of Src inhibitor PP1 (1.5 mg/kg in PBS/DMSO; BIOMOL Research Laboratories, Plymouth Meeting, Pa.) 5 minutes prior to injection with 100 μl of VEGF or bFGF (0.2 mg/kg in PBS; PeproTech, Rocky Hill, N.J.). At the appropriate time, the heart was rapidly excised and homogenized in 3 ml RIPA lysis buffer as previously described (Eliceiri et al. Mol. Cell 4: 915-924 (1999)) and the protein concentration measured (BCA Protein Assay; Pierce, Rockford, Ill.).

Ultrastructural Analysis by Electron Microscopy.

Cardiac tissue was prepared from mice following VEGF injection or 3-24 hours following ischemia and the infarct, the peri-infarct, and remote regions were sectioned. Tissue was fixed in 0.1 M sodium cacodylate buffer (pH 7.3) containing 4% paraformaldehyde+1.5% glutaraldehyde for 2 hours, transferred to 5% glutaraldehyde overnight, then 1% osmium tetroxide for 1 hour. Blocks were washed, dehydrated, cleared in propylene oxide, infiltrated with Epon/Araldite, and embedded in resin. Ultrathin sections were stained with uranyl acetate and lead citrate, and viewed using a Philips CM-100 transmission electron microscope.

Immunoprecipitation and Immunoblotting.

Tissue lysates were prepared for immunoprecipitation and immunoblotting as previously described (Eliceiri et al. Mol. Cell 4: 915-924 (1999)) with antibodies from Santa Cruz Biotechnology (Santa Cruz, Calif.): Flk (sc315), VE-cadherin (sc6458), β-catenin (sc7963), and P-Tyrosine (sc7020 or sc508). Representative data from at least three separate experiments is shown.

Statistical Analysis.

Data is presented as mean±standard error, with statistical significance determined from Student's t-test (P<0.05).

### Example 1

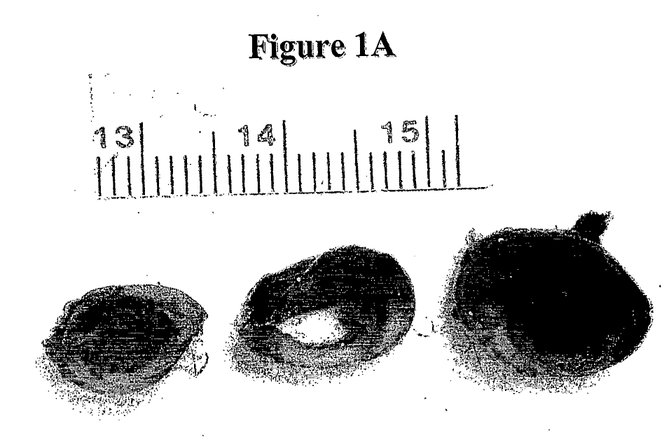

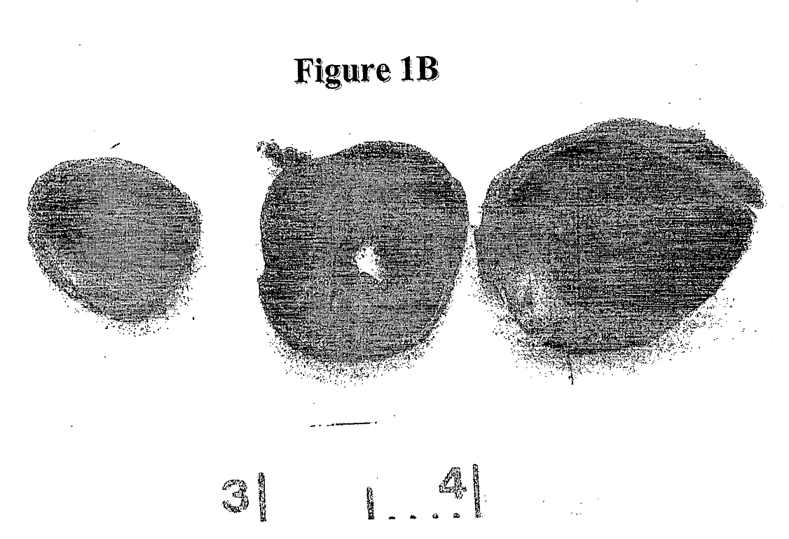

Blockade of Src activity resulted in cardioprotection, as shown by comparison of cardiac samples from the control subjects in FIG. 1A with those of the PP1-treated subjects in FIG. 1B.

### Example 2

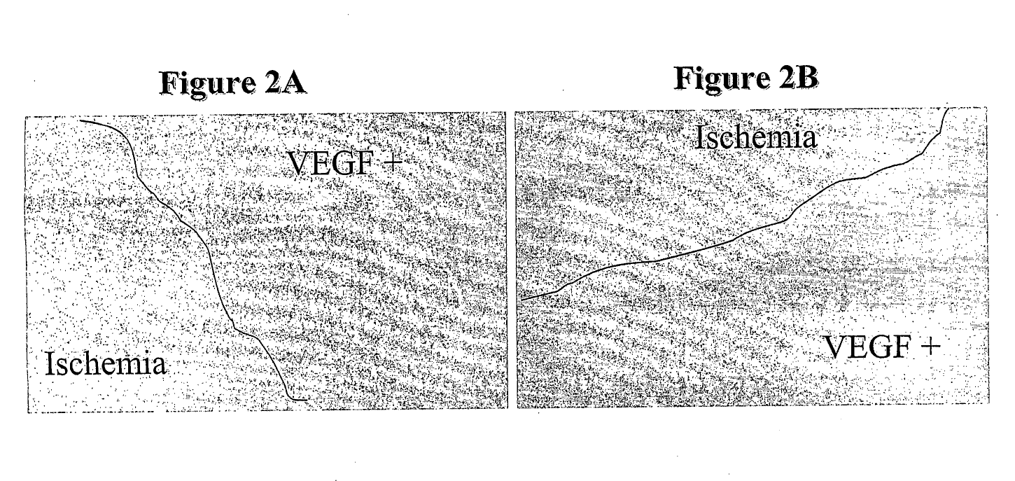

Src inhibition did not interfere with VEGF expression in the ischemic tissues. FIGS. 2A and 2B show the results of an immunohistochemistry assay for VEGF on rat heart samples 24 hours after induction of myocardial infarction, with VEGF+ and ischemic regions indicated. FIG. 2A shows the results in control rat cardiac tissue, while FIG. 2B shows the results in PP1-treated rat cardiac tissue.

### Example 3

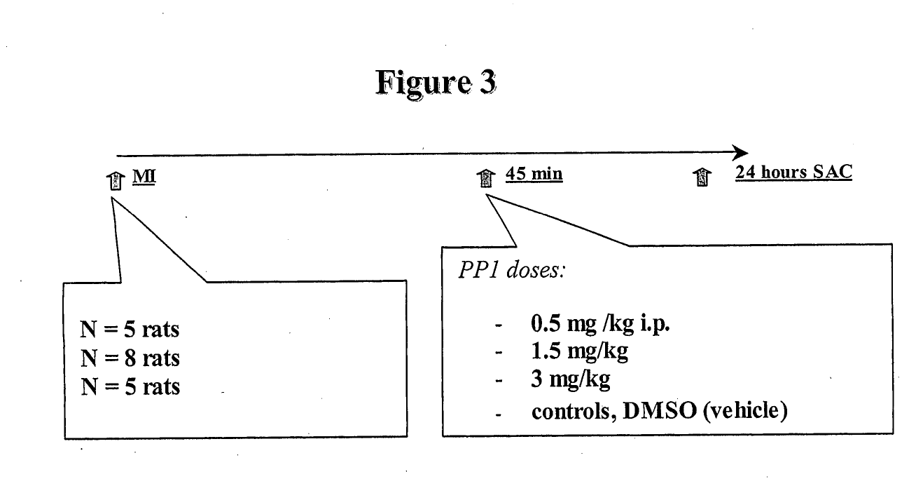

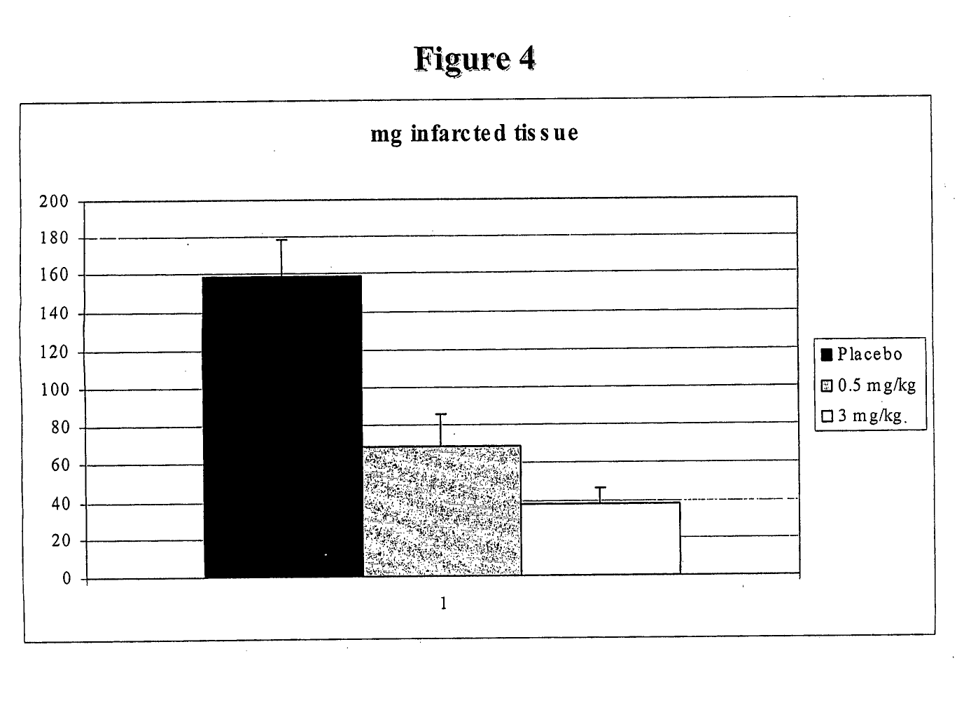

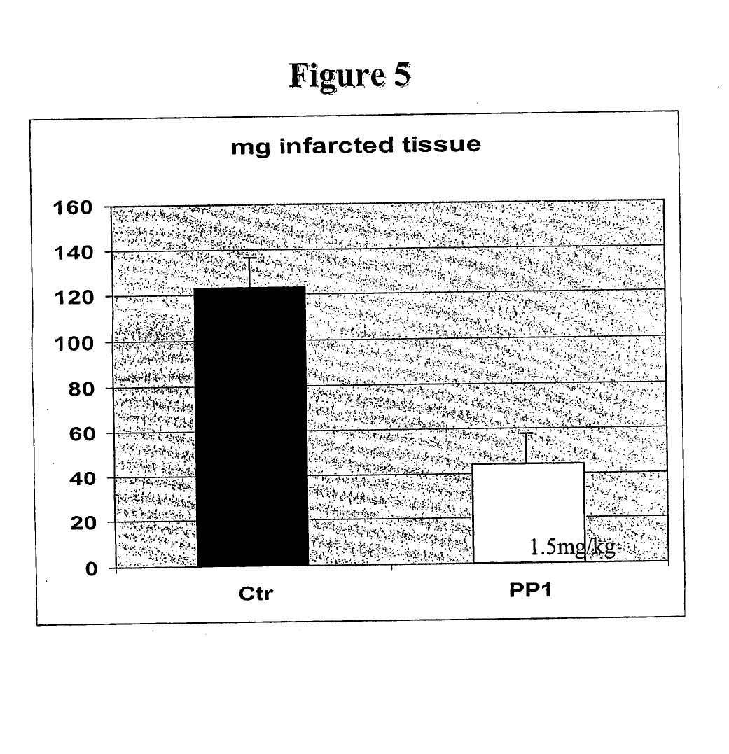

FIG. 3 is a schematic of the protocol used to measure the dose-dependent effect of PP1 on infarct size. FIG. 4 is a graph showing dose-dependent reduction of MI size by PP1. FIG. 5 is a graph showing the maximum dosage effects of Src deficiency and blockade on myocardial ischemia.

Essentially, MI was induced in rats as described above. As shown in FIG. 3, 45 min after MI induction, three groups of rats were treated with intraperitoneal injections of PP1: 0.5 mg/kg (5 rats), 1.5 mg/kg (8 rats), or 3 mg/kg (5. rats). Control rats were mock-treated with the dimethylsulfoxide (DMSO) vehicle. Tests were performed 24 hours post-MI induction.

As shown in FIGS. 4 and 5, Src inhibition decreased infarct size and area at risk in a dose-dependent manner 24 hours post-MI. A maximum inhibition of 68% (p<0.05) in infarct size was achieved at 1.5 mg/kg Src-inhibitor delivered 45 minutes after MI induction (FIG. 5).

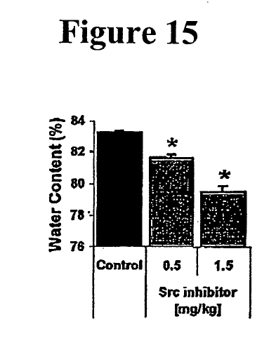

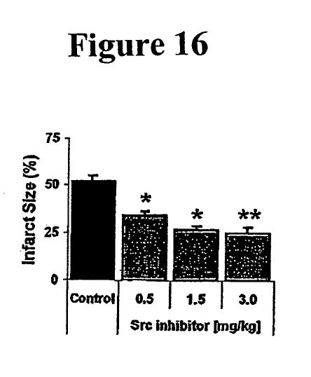

Additional experiments showed that PP1 provided dose-dependent decreases in edema and infarct size, with a maximum decrease at 1.5 mg/kg (n>5 each group, P<0.001) (FIGS. 15 and 16). PP1 also provided significant reduction of infarct size when administered following permanent occlusion in the mouse and rat.

### Example 4

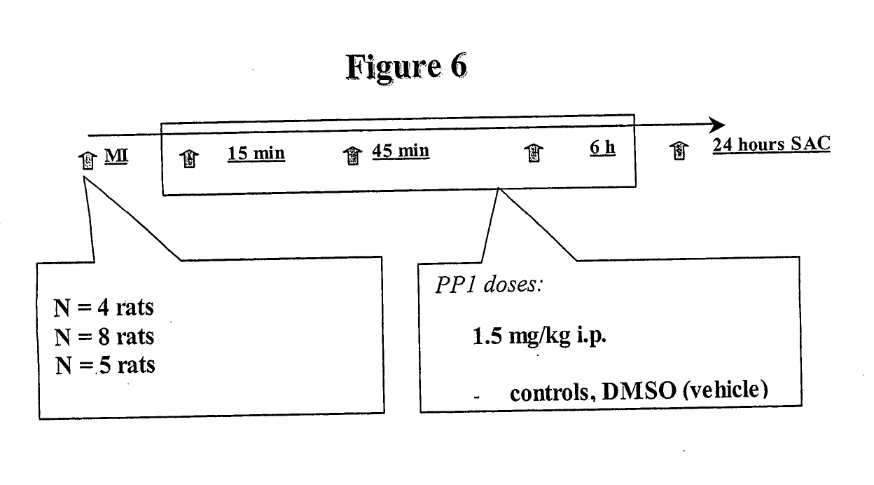

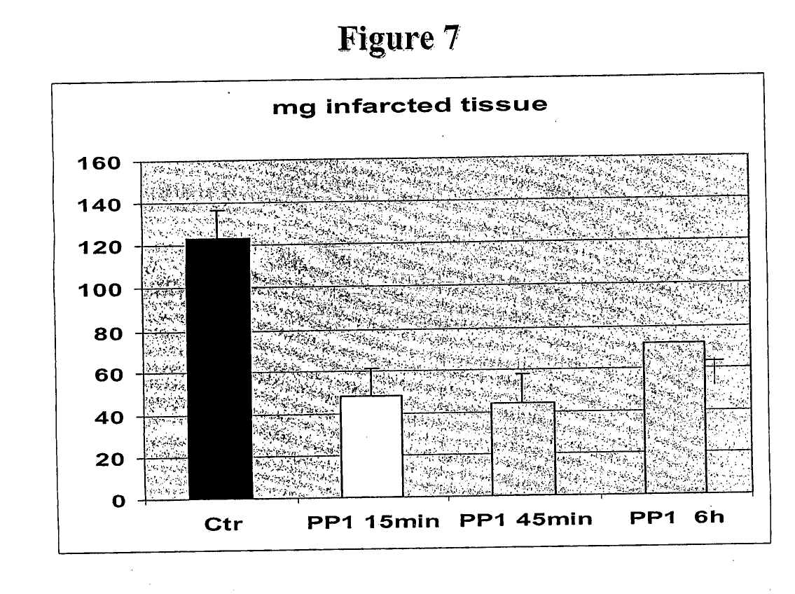

FIG. 6 is a schematic of the protocol used to measure the PP1-dependent decrease of infact size six hours after ischemia. FIG. 7 is a graph showing the effects of Src deficiency and blockade on myocardial ischemia in a murine model.

To study the kinetics of this response, PP1 was administered at various times following occlusion. Essentially, MI was induced in rats as described above. As shown in FIG. 6, 1.5 mg/g was administered via intraperitoneal injection to-three groups of rats 15 min (4 rats), 45 min (8 rats), or 6 hours (5 rats) post-MI induction. Control rats were mock-treated with the dimethylsulfoxide (DMSO) vehicle. Tests were performed 24 hours post-MI induction.

As shown in FIG. 7, PP1 was effective not only when administered 15 min or 45 min post-MI induction, but also when given six hours after LAD ligation resulting in a 42% decrease (p<0.05) in infarct size.

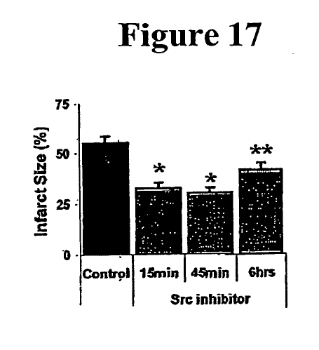

Additional experiments showed that, while maximum benefit (50% smaller infarct size) was achieved with administration 45 minutes following occlusion, treatment after 6 hours still yielded 25% protection (n=5 each group, P<0.05) (FIG. 17).

### Example 5

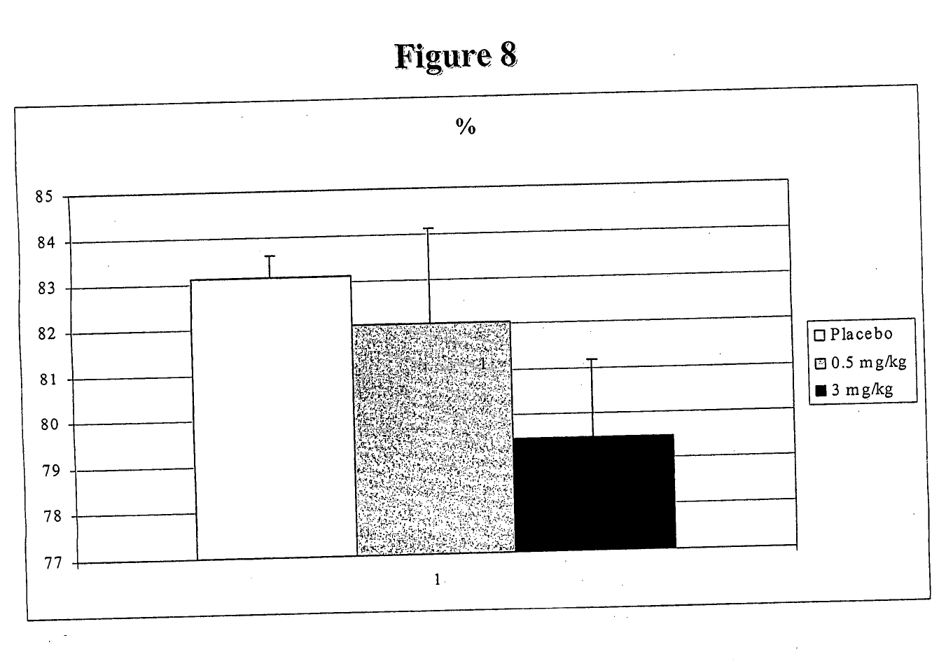

FIG. 8 is a graph showing the effects of PP1 treatment resulting in reduced infarct size accompanied by decreased myocardial water content. FIG. 9 is a photograph of in vivo magnetic resonance imaging showing the reduction in volume of edematous tissue.

Because of their increased water content edematous regions are expected to have a longer 72 relaxation than nonedematous regions. As a result, T2 maps of the myocardium can be used as an index of water content. Regions with T2>40 ms (two standard deviations above normally perfused myocardium) were delineated as an index of edema. This study showed a difference between LV volumes with T2>40 ms between Src inhibitor PP1 treated and control rats.

Rats were treated with 0.5 mg/kg, 1.5 mg/kg, or a placebo post-MI induction and the myocardial water content was compared. As shown in FIG. 8, reduced infarct size was accompanied by decreased myocardial water content (5%+/−1.3; p<0.05) and reduction in volume of the edematous tissue as detected by MRI (FIG. 9), indicating that the beneficial effect of Src inhibition was associated with prevention of VEGF-mediated VP. Similar results have been achieved using SKI-606 treated rats.

### Example 6

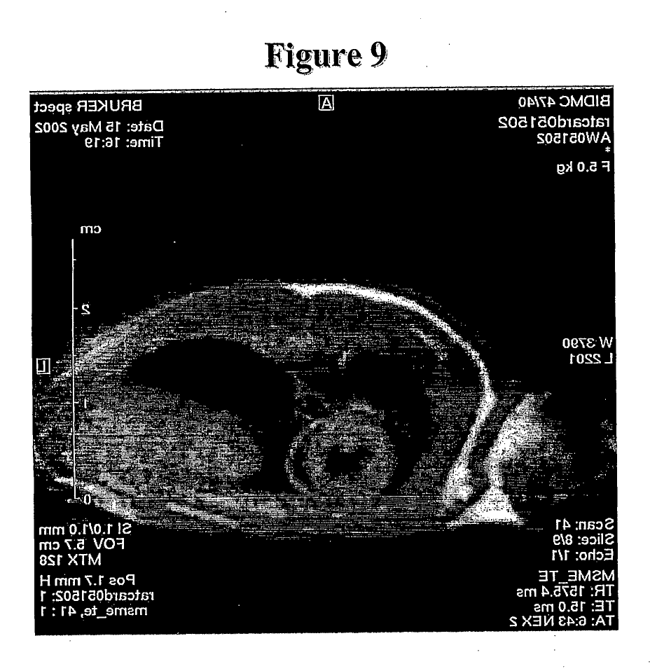

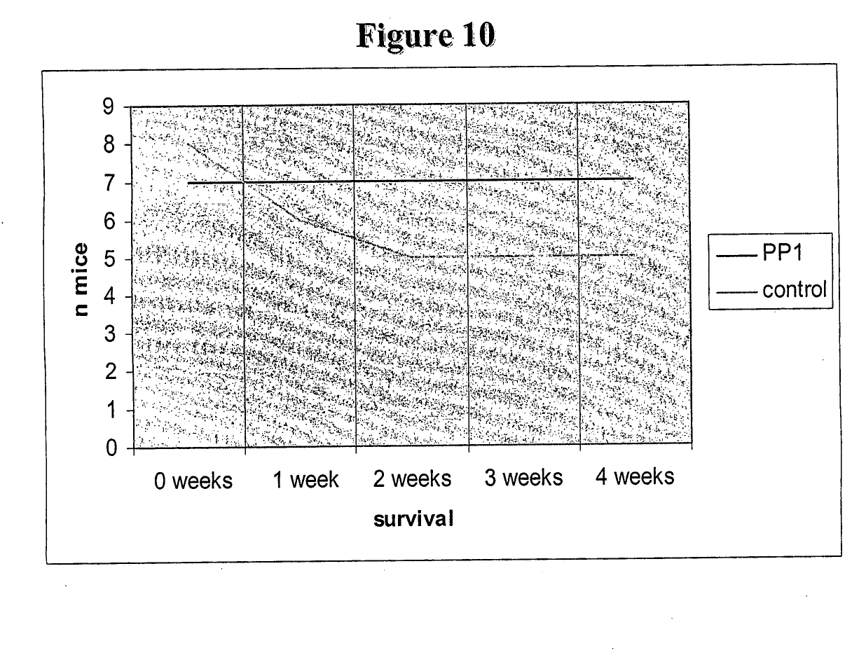

FIG. 10 is a graph showing the four-week survival rate for PP1-treated (1.5 mg/kg) and control mice.

MI was induced in mice. 1.5 mg/kg PP1 was administered to the experimental group of mice. Survival rates were assessed.

To evaluate survival after MI, 2-year-old C57 black mice were used as a model characterized by considerable mortality (>40%) after LAD ligation. Administration of PP1 (1.5 mg/kg) 45 minutes post-MI increased survival compared with control within the first 4 weeks (91.7% vs. 58.3%, respectively, n=12 each group), demonstrating a long-term therapeutic effect of Src inhibition.

Most important, four-week survival rate was 100% for treated and 62.5% for control mice, as shown in FIG. 10.

### Example 7

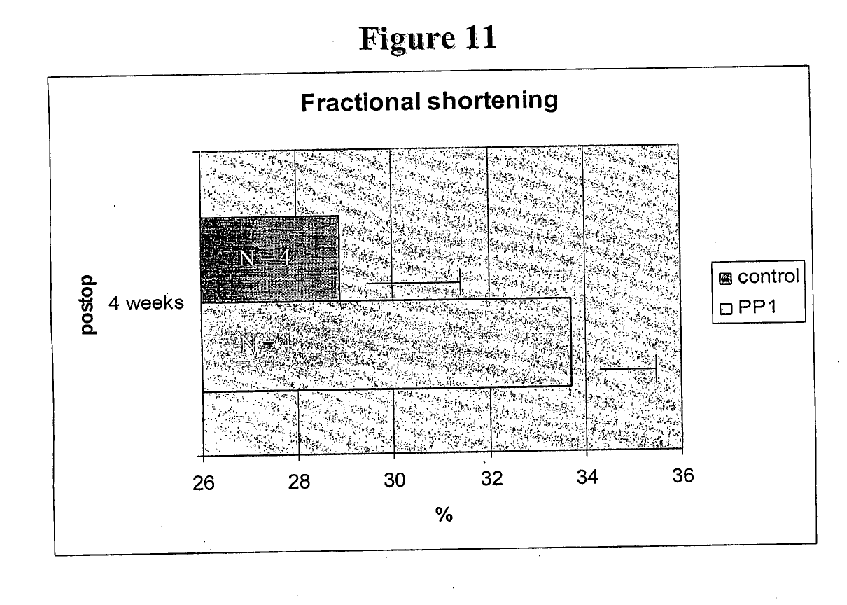

FIG. 11 is a graph showing the results of echocardiography testing on PP1-treated and control rats (4 weeks post-operative).

MI was induced in rats as described above. 1.5 mg/kg was administered via intraperitoneal injection to the experimental group of rats (4 rats), but not to the control rats (4 rats). Tests were performed 24 hours post-MI induction. Four weeks post-MI, fraction shortening was assessed by echocardiography.

As shown in FIG. 11, fractional shortening assessed by echocardiography 4 weeks post-MI was 28.9% in control and 33.7% in treated rats (p<0.05).

Additional echocardiography revealed Src inhibition with PP1 offers significant-preservation of fractional shortening (46%, n=8 -each group, P<0.05) and diastolic left ventricular diameter (11%, n=8, P<0.05) over 4 weeks compared with untreated rats, indicating that contractile function in the rescued tissue was preserved long term. Src inhibition also provided a favorable effect on systolic LV diameter (16%, n=8, P<0.05) and regional wall motion (9%, n=8, P<0.05).

Similar results have been achieved using SKI-606 treated rats. Treatment with the SKI-606 Src inhibitor also favorably impacted fractional shortening and regional wall motion score after 24 hours (n=7 each group, P<0.01).

### Example 8

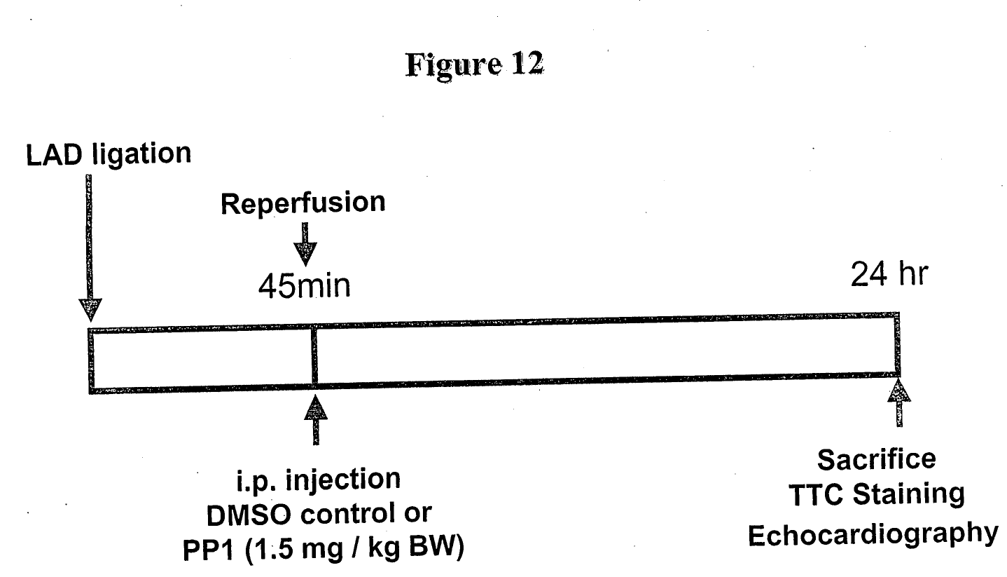

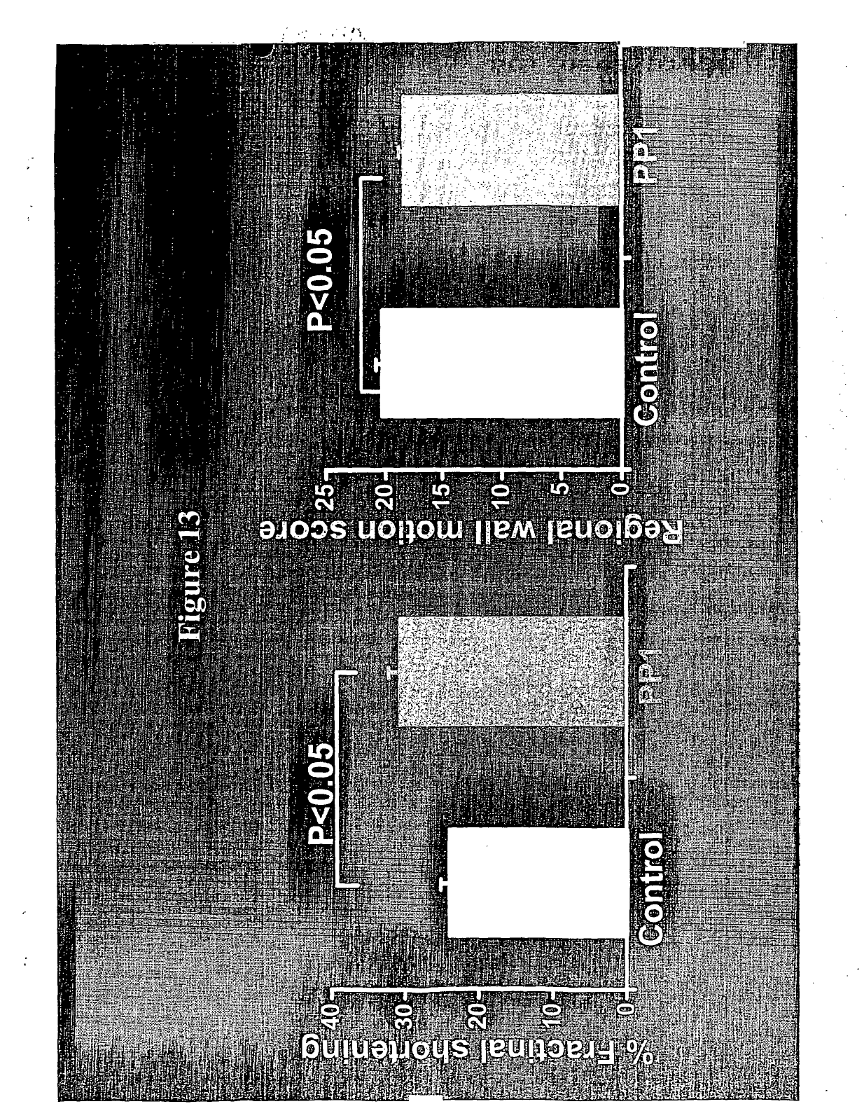

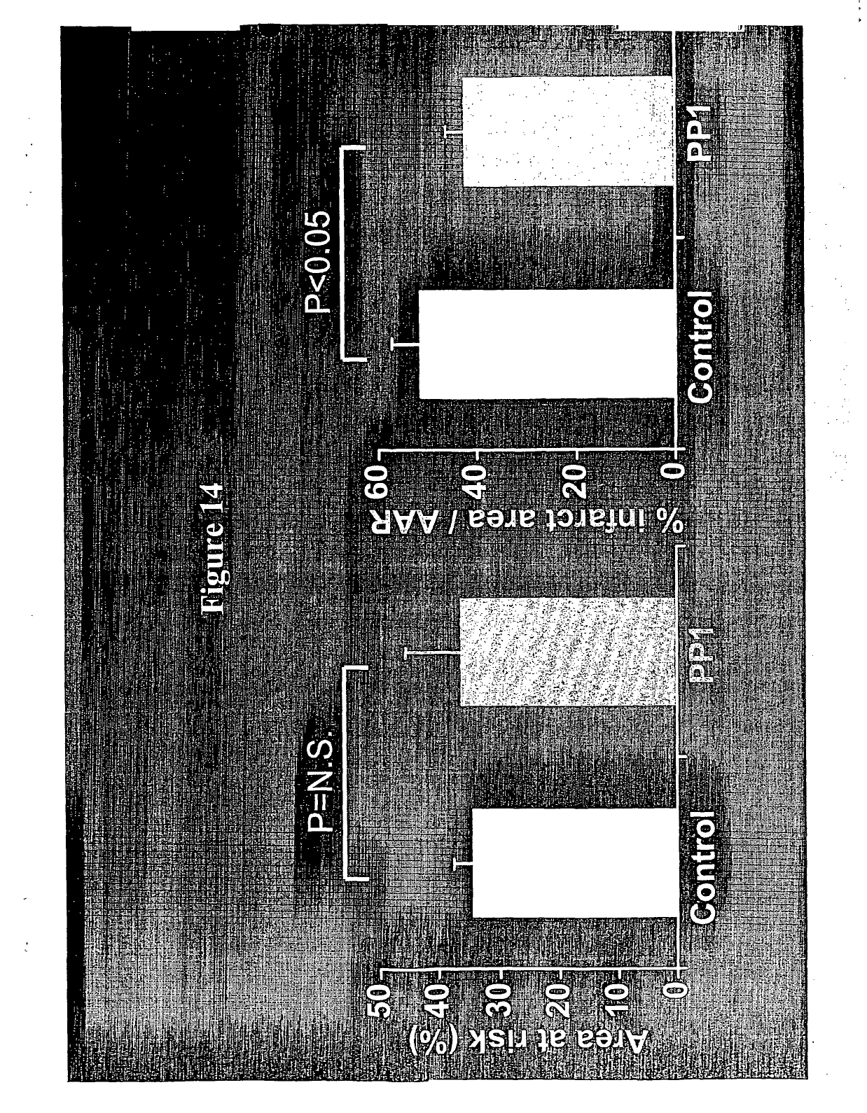

FIG. 12 is a schematic of the protocol used to measure the PP1-dependent decrease of ischemia/reperfusion in rats during a 24-hour period. FIG. 13 is a comparison of two graphs showing the results of echocardiography testing on treated and control rats. FIG. 14 is a comparison of two graphs showing the results of Evan's blue and TTC-staining on treated and control rats.

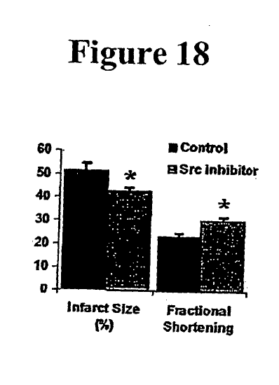

To establish whether Src inhibition is beneficial following transient ischemia, rats were subjected to occlusion followed by reperfusion, and then evaluated for ventricular function and infarct size after 24 hours. LAD ligation was performed on male Sprague-Dawley Rats (age 6-8 weeks), followed by reperfusion and intraperitoneal injection of DMSO (control rats) or 1.5 mg/kg PP1 (treated rats). Subjects were evaluated with echocardiography (FIGS. 13 and 18) and TTC staining (FIGS. 14 and 18) 24 hours after occlusion (Table 7).

Results

TABLE 7
Reperfusion Studies with PP1.

| No. | Drug | LVDd | LVDs | % FS | RWMS | AAR | IA/AAR |
|---|---|---|---|---|---|---|---|
| 1 | Control | 0.737 | 0.559 | 24.1 | 21 | 36.5 | 53.6 |
| 2 | Control | 0.856 | 0.678 | 20.8 | 20 | 33.2 | 55.1 |
| 3 | PP1 | 0.877 | 0.633 | 27.8 | 18 | 34.4 | 47 |
| 4 | Control | 0.822 | 0.627 | 23.7 | 21 | 29.9 | 42.7 |
| 5 | PP1 | 0.797 | 0.551 | 30.9 | 19 | 49.7 | 37.9 |
| 6 | PP1 | 0.729 | 0.475 | 34.9 | 18 | 32.9 | 42.1 |
| 7 | PP1 | 0.737 | 0.525 | 28.7 | 19 | 27.2 | 42.4 |
| 8 | Control | 0.737 | 0.542 | 26.4 | 19 | 37.2 | 54.6 |

Echocardiography
% FS: Control: 23.8±2.3 v.s. PP1: 30.6±3.16 (p<0.05 (0.013))
RWMS: Control: 20.3±0.96 v.s. PP1: 18.5±0.58 (p<0.05 (0.0203))
   Evan's Blue & TTC Staining
Area at risk: Control: 34.2±3.36 v.s. PP1: 36.1±9.61 (p=N.S. (0.7310))
% infarct/AAR (% of AAR): Control: 51.5±5.9 v.s. PP1: 42.4±3.72 (p<0.05 (0.0397))

Src inhibition by PP1 preserved LV fractional shortening (FIG. 13) and reduced infarct size (FIG. 14) compared to controls (n=4 each group, P<0.05) (see also FIG. 18). The 18% reduction in infarct size following ischemia-reperfusion (FIG. 14) compares to a 50% decrease following permanent occlusion in which the hypoxic stimulus driving VEGF expression is maintained.

### Example 9

The protocols outlined in Example 8 were repeated with SKI-606 inhibitor (Src-I) (Wyeth-Ayerst Research) (see Tables 8 and 9).

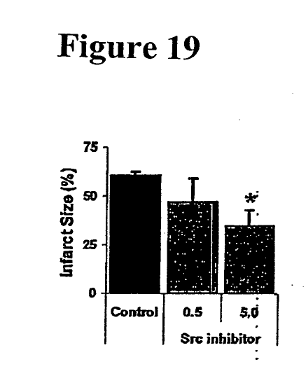

SKI-606 (5 mg/kg) provided a 43% decrease in infarct size in the ischemia-reperfusion model (n5 each group, P<0.01) (FIG. 19). Together with the data on PP1 in the above examples, this data supports a beneficial effect of Src inhibition following transient ischemia.

Results

TABLE 8
Reperfusion Studies with SKI-606.

| | | | | | | RW | wet | dry | % | | | (%) | (%) |
|---|---|---|---|---|---|---|---|---|---|---|---|---|---|
| No. | B.W. | Drug | Dd | Ds | % FS | MS | weight | weight | water | AAR | IA/AAR | T2 > 40 | T2 > 35 |
| 1 | 175 | Src-I | 0.669 | 0.513 | 23.3 | 21 | 0.64 | 0.15 | 76.6 | 42.3 | 52.4 | 19.466 | 28.257 |
| 2 | 180 | Control | 0.801 | 0.623 | 22.2 | 21 | 0.67 | 0.14 | 79.1 | 44.6 | 59.5 | 21.847 | 32.106 |
| 3 | 225 | Src-I | 0.89 | 0.669 | 24.8 | 20 | 0.66 | 0.16 | 75.8 | 36.5 | 53 | 5.719 | 9.198 |
| 4 | 215 | Control | 0.847 | 0.669 | 21 | 23 | 0.71 | 0.15 | 78.9 | 39.7 | 52.4 | 18.111 | 29.402 |
| 5 | 200 | Src-I | 0.873 | 0.661 | 23.2 | 20 | 0.73 | 0.16 | 78 | 38.6 | 58.6 | 17.955 | 27.518 |
| 6 | 215 | Control | 0.847 | 0.678 | 20 | 24 | 0.75 | 0.16 | 78.7 | 33.5 | 65.6 | 23.372 | 31.59 |
| 7 | 275 | Src-I | 0.881 | 0.669 | 24 | 21 | 0.78 | 0.2 | 74.4 | 35.2 | 55.5 | NO MRI | |
| 8 | 285 | Control | 0.814 | 0.653 | 19.8 | 23 | 0.93 | 0.2 | 78.5 | 33.3 | 62 | NO MRI | |
| 9 | 275 | Src-I | 0.703 | 0.669 | 24.1 | 21 | 0.85 | 0.22 | 74.1 | 36.7 | 54.5 | 5.743 | 13.699 |
| 10 | 275 | Control | 0.873 | 0.686 | 21.4 | 23 | 0.82 | 0.18 | 78 | 34.2 | 62 | NO MRI | |
| 11 | 270 | Control | 0.941 | 0.763 | 18.9 | 24 | 0.85 | 0.2 | 76.5 | 35.2 | 60.2 | NO MRI | |
| 12 | 200 | Control | 0.763 | 0.593 | 22.2 | 22 | 0.65 | 0.14 | 79.7 | | | NO MRI | |
| 13 | 205 | Src-I | 0.805 | 0.619 | 23.2 | 23 | 0.67 | 0.14 | 79.1 | | | NO MRI | |
| 14 | 220 | Control | 0.831 | 0.669 | 19.4 | 24 | 0.72 | 0.14 | 80.6 | | | 50.794 | 68.11 |
| 15 | 210 | Src-I | 0.865 | 0.678 | 21.6 | 23 | 0.75 | 0.16 | 78.7 | | | 7.537 | 13.287 |

TABLE 9
Summary of SKI-606 T2 > 40

| Group | T2 > 40 |
|---|---|
| Control | 21.847 |
| Control | 18.111 |
| Control | 23.372 |
| Control | 50.794 |
| Control | 23.653 |
| Src-I | 19.466 |
| Src-I | 5.719 |
| Src-I | 17.955 |
| Src-I | 5.743 |
| Src-I | 7.537 |

Echocardiography
% FS: Control: 20.6±1.27 vs Src-inhibitor: 23.5±1.01 (p<0.05 (0.0004))
RWMS: Control: 23.0±1.07 vs Src-inhibitor: 21.3±1.25 (p<0.05 (0.0134))
   Evan's Blue & TTC Staining
Area at Risk: Control: 36.8±4.51 v.s. Src-inbibitor: 37.9±2.76 (p=N.S. (0.7310))
% Infarct/AAR (% of AAR): Control: 60.3±4.4 v.s. Src-inhibitor: 54.8±2.45 (p<0.05 (0.0356))
   MRI
% 17>40 Control: 28.5±15 v.s. Src-inhibitor: 11.3±6.84 (p=N.S. (0.0537))
% T2>35 Control: 40.3±18.58 v.s. Src-inhibitor: 18.4±8.85 (p=N.S. (0.0508))
Water Content Control: 78.8±1.21 v.s. Src-inhibitor: 76.7±2.01 (p<0.05 (0.0283))

### Example 10

Previous in vitro studies have implicated VEGF in the regulation of VE-cadherin function (Esser et al. J. Cell Sci. 111: 1853-1865 (1998)).

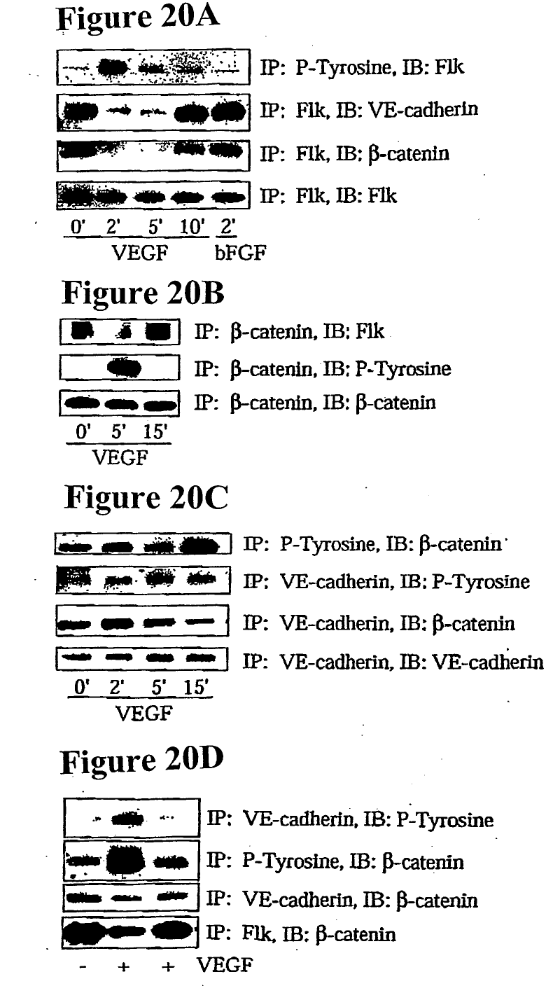

As shown in FIGS. 20A-20C, immnunoprecipitation (IP) and immunoblotting (IB) reveals a pre-formed Flk-cadherin-catenin complex which becomes phosphorylated and dissociates upon VEGF stimulation. As shown in FIG. 20D, Src is required for these VEGF-mediated signaling events, since the Flk-cadherin-catenin complex remains intact in mice pretreated with the Src inhibitor PP1 before VEGF injection. (Data is representative of at least three experiments.)

Heart lysates prepared from animals injected with or without VEGF were subjected to immunoprecipitation with anti-Flk followed by immunoblotting for VE-cadherin and β-catenin. In control mice, a pre-existing complex between Flk, β-catenin, and VE-cadherin in blood vessels was observed. This complex was rapidly disrupted within 2-5 minutes following VEGF stimulation, and had reassembled by 15 minutes in blood vessels in vivo. The timescale of complex dissociation completely paralleled that of Flk, β-catenin, and VE-cadherin phosphorylation and the dissociation of β-catenin from VE-cadherin. These VEGF-mediated events were Src-dependent since the Flk-cadherin-catenin signaling complex remained intact and phosphorylation of β-catenin and VE-cadherin did not occur in VEGF-stimulated mice pretreated with Src inhibitors. These events were not observed following injection of basic fibroblast growth factor (bFGF), a similar angiogenic growth factor which does not promote vascular permeability.

As shown in FIG. 20,

While a single VEGF injection produced a reversible, rapid, and transient signaling response which returned to baseline by 15 minutes, four VEGF injections (every thirty minutes) produced a prolonged signaling response. For example, dissociation of Flk-catenin and Erk phosphorylation persisted following prolonged VEGF exposure. This model may be applicable to the physiological situation following MI, where VEGF expression is prolonged as a result of ongoing tissue hypoxia.

### ADDITIONAL WORK

The following additional experiments are illustrative of the present invention:

Src blockade reduces edema and provides protection following MI

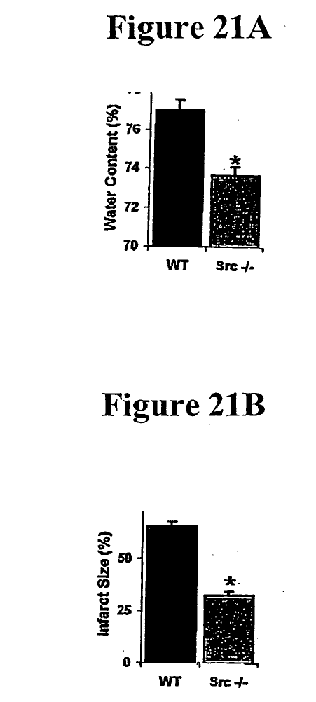

To establish the potential role of Src in the pathophysiology following MI, the effects of Src deletion on the murine heart were investigated following ligation of the left anterior descending (LAD) coronary artery. Twenty-four hours after the onset of ischemia, pp60Src^{-/-} mice had significantly decreased myocardial water content (P<0.01) associated with 50% smaller infarct size compared with heterozygous controls (n=4 each group, P<0.001) (FIGS. 21A and 21B). pp60Src^{+/-} mice show a normal permeability and signaling response to VEGF (Eliceiri et al. Mol. Cell 4: 915-924 (1999)). VEGF expression following MI was comparable between genotypes, demonstrating Src inhibition did not interfere with induction of VEGF, but rather influenced a downstream effector.

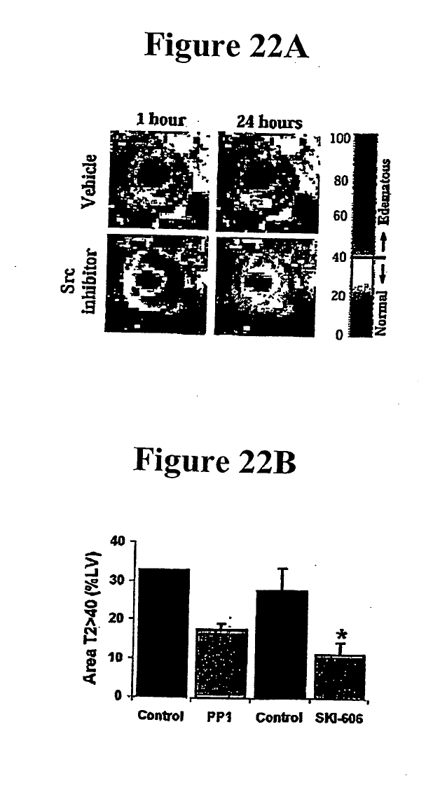

As a means of determining the potential for MRI to detect the spatial distribution of edematous regions of myocardium with Src inhibitor PP1 treatment (n=2), Src inhibitor SKI-606 treatment (n=5), and vehicle treatment (n=5), short axis maps of the MRI parameter T2 of the left ventricle (LV) were obtained 24 hours following permanent LAD occlusion in rats. Because of their increased water content, edematous regions are expected to have a longer T2 relaxation than nonedematous regions and therefore T2 maps of the myocardium can be used as an index of water content. Regions with T2>40 ms (two standard deviations above normally perfused myocardium) were delineated as an index of edema. Initial studies indicated a difference between LV volumes with T2>40 ms between Src inhibitor PP1 treated and vehicle treated rats (FIGS. 22A and 22B). The SKI-606 treated rats, as a percentage of total LV volume, had a mean T2>40 ms volume of 11.3±6.8% whereas vehicle treated rats had a mean T2>40 ms volume of 27.6±13.2% (P<0.05) showing the potential for MRI to be used as a noninvasive assessment of Src inhibitor treatment in vivo. Myocardial water content was also computed ex-vivo using wet/dry weights of nonischemic myocardium.

Chronic myocardial fibrosis occurs following infarction and is a direct reflection of the extent of tissue necrosis following MI. To evaluate the effect of Src inhibition on fibrosis 4 weeks post-MI in rats, histopathological analysis of fibrotic tissue was performed using elastic trichrome staining. Src inhibition contributed to a 52% decrease in LV fibrotic tissue compared with control (19.1±2.2% vs. 40.0±3.0%, n=4 each group, P<0.01). Better preservation of myocardial fibers and LV architecture were consistently observed among the samples which received the Src inhibitor, indicating that Src inhibition contributes to a long term protective effect on the myocardium post-MI.

Effect of MI on vascular integrity and myocyte viability in the peri-infarct zone

Since VEGF expression increases primarily in the peri-infarct zone, the ultrastructural effects of Src inhibition on small vessels in this region were investigated 3-24 hours post-MI. In contrast to normal myocardial tissue, numerous examples of damage were observed in the peri-infarct zone. Extravasated blood cells (RBC, platelets, and neutrophils) were present in the interstitium, apparently escaped from nearby vessels. Some EC were swollen and occluded part of the vessel lumen, often appearing electron-lucent and containing many caveolae. Large round vacuoles were present in the endothelium, often several times larger than the EC thickness. Myocyte injury increased with time following MI and varied between adjacent cells, identifiable as mitochondrial rupture, disordered mitochondrial cristae, intracellular edema, and myofilament disintegration. The most affected myocytes were often adjacent to injured blood vessels or free blood cells. Neutrophils, which participate in the acute response to injury, were frequently observed 24 hours after MI.

Accumulation of microthrombi in EC gaps

Three hours following MI, gaps between adjacent EC were frequently observed. Surprisingly, many of the gaps observed were plugged by platelets. Some platelets contacted the basal lamina exposed between EC, while in other cases the basal lamina also appeared to be disrupted. Some of the platelets were degranulated and may potentiate the further activation, adhesion, and aggregation of circulating platelets. While these platelet plugs may prevent further vascular leak, they could inadvertently contribute to decreased perfusion in small vessels via microthrombi formation and lead to further ischemia-related tissue disease.

Src blockade prevents VP and myocyte damage

To test whether Src inhibition could block microvascular hyperpermeability at the ultrastructural level, animals were treated with PP1 (1.5 mg/kg) or vehicle 45 minutes following coronary artery occlusion. Src inhibition dramatically protected the peri-infarct region from endothelial barrier dysfunction and blood vessel damage (Table 1). The most notable was the impact of PP1 at 24 hours, revealing a significant reduction in myocyte injury. While PP1 did not abrogate all evidence of damage, it did prevent vascular gaps and resulted in a vastly improved EC ultrastructural appearance, and provided protection to the blood vessels and myocytes. These results provide an ultrastructural basis for the improvement in ventricular function and survival measured at 24 hours post-MI in the animals receiving the Src inhibitor.

MI and systemic VEGF injection produce a similar vascular response

To determine the contribution of VEGF to this complex pathology, the growth factor was injected intravenously into normal mice and evaluated cardiac tissue at the ultrastructural level after 30 minutes. Surprisingly, the extent of VEGF-induced endothelial barrier dysfunction and vessel injury was comparable to that seen in the peri-infarct zone post-MI. Considerable platelet adhesion to the EC basement membrane as well as myocyte damage was observed. Similar evidence of damage was found in the brain following systemic VEGF injection, suggesting these effects may be systemic.

To determine whether VEGF is sufficient to mediate longer term pathology associated with MI, mice were injected four times with VEGF over the course of 2 hours. This treatment created damage similar to that observed 24 hours post-MI. Platelet adhesion, neutrophils, and significant myocyte damage, as well as numerous electron-lucent EC, many of which were swollen to occlude the vessel lumen. Taken together, 30 minutes exposure to VEGF is sufficient to induce a similar ultrastructure observed after 3 hours of MI, by which time VEGF expression is significantly increased in the peri-infarct zone. However, longer term VEGF exposure elicited vascular remodeling similar to that seen in tissues 24 hours after MI.

No signs of a vascular response following VEGF injection were seen in the pp60Src^{-/-} animal (Table 1), compared with gaps, platelet activity, affected EC, and extravasated blood cells in wildtype mice. The complete blockade of any response suggests that VEGF-mediated Src activity initiates a cascade leading to VP-induced injury during ischemic disease.

TABLE 1
Ultrastructural observations in mouse cardiac
tissue following MI or VEGF injection

| | EC | Platelet | | |
|---|---|---|---|---|
| | Barrier | Activation | EC | Cardiac |
| | Dysfunction | & Adhesion | Injury | Damage |
| 3 hr MI | 18 | 36 | 31 | 22 |
| 3 hr MI + PP1 | 2 | 11 | 14 | 2 |
| 24 hr MI | 5 | 7 | 34 | 45 |
| 24 hr MI + PP1 | 0 | 1 | 15 | 9 |
| Control | 0 | 0 | 1 | 0 |
| VEGF, pp60Src^{+/+} | 24 | 18 | 33 | 16 |
| VEGF, pp60Src^{-/-} | 0 | 0 | 0 | 0 |

For each group, left ventricular tissue was examined for 4 hours (approximately 250 microvessels) on a transmission electron microscope and observations were counted and grouped according to:
EC Barrier Dysfunction: Gaps, Fenestrations, Extravasated blood cells
Platelet Activation/Adhesion: Platelets, Degranulated platelets, Platelet clusters, Platelet adhesion to ECM
EC Injury: Electron-lucent EC, Swollen EC, Large EC vacuoles, Occluded vessel lumen
Cardiac Damage: Mitochondrial swelling, Disordered cristae, Myofilament disintegration

### CONCLUSIONS

The Examples show that two structurally distinct Src inhibitors produce the same effect as seen in Src-deficient mice indicating the role of Src in the pathology related to VP-associated tissue injury following MI. Essentially identical Src-dependent ultrastructural changes were observed following MI or direct VEGF injection. Moreover, most of the changes observed were directly associated with changes in EC cell-cell contact and blood vessel integrity, none or few of which were seen in either Src knockout animals or wild type animals treated with Src inhibitors.

Throughout this application, various publications are referenced by author and year and patents by number. The disclosures of these publications and patents in their entireties are hereby incorporated by reference into this application in order to describe more fully the state of the art to which this invention pertains.

The invention has been described in an illustrative manner, and it is to be understood that the terminology which has been used is intended to be in the nature of words or description, rather than of limitation.

Many modifications and variations of the present invention are possible in light of the above teachings. It is, therefore, to be understood that within the scope of the described invention, the invention may be practiced otherwise than as specifically described.

### REFERENCES

1. Paul, R., Z. G. Zhang, B. P. Eliceiri, Q. Jiang, A. D. Boccia, R. L. Zhang, M. Chopp & D. A. Cheresh (February 2001), “Src deficiency or blockade of Src activity in mice provides cerebral protection following stroke, ” Nature Medicine 7(2):222-227.
2. Schlessinger, J. (Feb. 4, 2000), “New roles for Src kinases in control of cell survival and angiogenesis,” Cell 100(3):293-296.
3. Eliceiri, B. P., R. Paul, P. L. Schwartzberg, J. D. Hood, J. Leng & D. A. Cheresh (December 1999), “Selective requirement for Src kinases during VEGF-induced angiogenesis and vascular permeability,” Mol. Cell 4(6):915-924.
4. Garcia-Dorado, D. & Oliveras, J. Myocardial oedema: a preventable cause of reperfusion injury? Cardiovasc Res 27, 1555-63 (1993).
5. Li, J. et al. VEGF, flk-1, and flt-1 expression in a rat myocardial infarction model of angiogenesis. Am J Physiol 270, H1803-11 (1996).
6. Esser, S., Lampugnani, M. G., Corada, M., Dejana, E. & Risau, W. Vascular endothelial growth factor induces VE-cadherin tyrosine phosphorylation in endothelial cells. J Cell Sci 111, 1853-65 (1998).
7. Feng, D., Nagy, J. A., Hipp, J., Dvorak, H. F. & Dvorak, A. M. Vesiculo-vacuolar organelles and the regulation of venule permeability to macromolecules by vascular permeability factor, histamine, and serotonin. J Exp Med 183, 1981-6. (1996).
8. Roberts, W. G. & Palade, G. E. Increased microvascular permeability and endotheliai fenestration induced by vascular endothelial growth factor. J Cell Sci 108, 2369-79 (1995).
9. Neufeld, G., Cohen, T., Gengrinovitch, S. & Poltorak, Z. Vascular endothelial growth factor (VEGF) and its receptors. Faseb J 13, 9-22 (1999).
10. Carmeliet, P. et al. Targeted deficiency or cytosolic truncation of the VE-cadherin gene in mice impairs VEGF-mediated endothelial survival and angiogenesis. Cell 98, 147-57 (1999).
11. Xu, Y. & Carpenter, G. Identification of cadherin tyrosine residues that are phosphorylated and mediate Shc association. J Cell Biochem 75, 264-71. (1999).
12. Waltenberger, J., Claesson-Welsh, L., Siegbahn, A., Shibuya, M. & Heldin, C. H. Different signal transduction properties of KDR and Flt1, two receptors for vascular endothelial growth factor. J Biol Chem 269, 26988-95. (1994).
13. Hanke, J. H. et al. Discovery of a novel, potent, and Src family-selective tyrosine kinase inhibitor. Study of Lck- and FynT-dependent T cell activation. J Biol Chem 271, 695-701 (1996).
14. Fishbein, M. C. et al. Early phase acute myocardial infarct size quantification: validation of the triphenyl tetrazolium chloride tissue enzyme staining technique. Am Heart J 101, 593-600 (1981).
15. Schiller, N. B. et al. Recommendations for quantitation of the left ventricle by two-dimensional echocardiography. American Society of Echocardiography Committee on Standards, Subcommittee on Quantitation of Two-Dimensional Echocardiograms. J Am Soc Echocardiogr 2, 358-67 (1989).
16. Boschelli, D. H. et al. Synthesis and Src kinase inhibitory activity of a series of 4-phenylamino-3-quinolinecarbonitriles. J Med Chem.44(5): 822-33 (1 Mar. 2001).
17. Wang, Y. D. et al. Inhibitors of src tyrosine kinase: the preparation and structure-activity relationship of 4-anilino-3-cyanoquinolines and 4-anilinoquinazolines. Bioorg Med Chem Lett. 10(21):2477-80 (6 Nov. 2000).

What is claimed is:

1. A method for treating, preventing, or reducing reperfusion injury or post-pump syndrome by administering an inhibitor of vascular endothelial growth factor-mediated vascular permeability.

2. The method of claim 1, wherein the inhibitor comprises an inhibitor of a Src family kinase.

3. The method of claim 1, wherein the inhibitor comprises a pyrazolopyrimidin.

4. The method of claim 1, wherein the inhibitor comprises PP1 or PP2.

5. The method of claim 1, wherein the inhibitor has the chemical formula C₁₆H₁₉N₅.

6. The method of claim 1, wherein the inhibitor comprises a quinolinecarbonitrile.

7. The method of claim 1, wherein the inhibitor comprises a 3-quinolinecarbonitrile.

8. The method of claim 1, wherein the inhibitor comprises a 4-anilino-3-quinolinecarbonitrile.

9. The method of claim 1, wherein the inhibitor comprises SKI-606.

10. The method of claim 2, wherein the Src family kinase comprises Src, Fyn, Yes, Lyn, Lck, or Hck.

11. The method of claim 2, wherein the Src family kinase comprises Src.

12. The method of claim 1, wherein the inhibitor is administered intravenously.

13. The method of claim 1, wherein the inhibitor is administered by intraperitoneal injection.

14. The method of claim 1, wherein the inhibitor is administered using an intracoronary method.

15. The method of claim 1, wherein the inhibitor is administered percutaneously.

16. The method of claim 1, wherein the reperfusion injury is the result of myocardial infarction.

17. The method of claim 1, wherein the reperfusion injury is the result of angina.

18. The method of claim 1, wherein the reperfusion injury or post-pump syndrome is the result of a coronary revascularization procedure.

19. The method of claim 18, wherein the coronary revascularization procedure comprises a percutaneous coronary revascularization procedure.

20. The method of claim 19, wherein the percutaneous coronary revascularization procedure comprises angioplasty, stent placement, or atherectomy.

21. The method of claim 18, wherein the coronary revascularization procedure comprises angioplasty, comprising an angioplasty balloon, wherein the balloon comprises a coating comprising an inhibitor of vascular endothelial growth factor-mediated vascular permeability.

22. The method of claim 21, wherein the inhibitor comprises an inhibitor of a Src family kinase.

23. The method of claim 18, wherein the coronary revascularization procedure comprises angioplasty, comprising an angioplasty balloon, wherein the angioplasty balloon is capable of eluting an inhibitor of vascular endothelial growth factor-mediated vascular permeability.

24. The method of claim 23, wherein the inhibitor comprises an inhibitor of a Src family kinase.

25. The method of claim 18, wherein the coronary revascularization procedure comprises stent placement, wherein the stent comprises a coating comprising an inhibitor of vascular endothelial growth factor-mediated vascular permeability.

26. The method of claim 25, wherein the inhibitor comprises an inhibitor of a Src family kinase.

27. The method of claim 18, wherein the coronary revascularization procedure comprises stent placement, wherein the stent is capable of eluting an inhibitor of vascular endothelial growth factor-mediated vascular permeability.

28. The method of claim 27, wherein the inhibitor comprises an inhibitor of a Src family kinase.

29. The method of claim 18, wherein the coronary revascularization procedure comprises a surgical coronary revascularization procedure.

30. The method of claim 29, wherein the surgical coronary revascularization procedure comprises bypass surgery.

31. The method of claim 1, wherein the reperfusion injury is the result of stroke or a treatment for stroke.

32. The method of claim 1, wherein the reperfusion injury is the result of compartment syndrome or a treatment for compartment syndrome.

33. A method for treating, preventing, or reducing reperfusion injury following ischemia, wherein the ischemia is caused by blockage or leakage of a blood vessel, by administering an inhibitor of vascular endothelial growth factor-mediated vascular permeability, wherein
a. the inhibitor comprises an inhibitor of a Src family kinase; and
b. the ischemia is the result of:
i. myocardial infarction;
ii. stroke;
iii. compartment syndrome;
iv. post-pump syndrome; or
v. angina.

34. The method of claim 33, wherein the Src family kinase comprises Src, Fyn, Yes, Lyn, Lck, or Hck.

35. The method of claim 33, wherein the inhibitor comprises a pyrazolopyrimidin or a 3quinolinecarbonitrile.

36. The method of claim 33, wherein the inhibitor comprises PP1, PP2, or SK1-606.

37. The method of claim 33, wherein the inhibitor is administered by intravenous, by intraperitoneal injection, by direct injection into an artery, by infusion, by an intracoronary method, or by percutaneous administration.

38. A method for treating, preventing, or reducing injury following bypass surgery by administering an inhibitor of vascular endothelial growth factor-mediated vascular permeability, wherein the inhibitor comprises an inhibitor of a Src family kinase.

39. The method of claim 38, wherein the inhibitor is administered as part of the cardioplegia solution.

40. A method for treating, preventing, or reducing reperfusion injury following compartment syndrome by administering an inhibitor of vascular endothelial growth factor-mediated vascular permeability, wherein the inhibitor comprises an inhibitor of a Src family kinase.

41. The method of claim 40, wherein the inhibitor is administered by infusion into a local artery during a surgical procedure for the treatment or relief of the compartment syndrome.
